(19) United States
(12) Patent Application Publication
(10) Pub. No.: US 2026/0198986 A1
(43) Pub. Date: Jul. 16, 2026

(54) TECHNOLOGIES FOR CONTROLLING A RADIO FREQUENCY MODE OF ENERGY-BASED SURGICAL INSTRUMENTS

(71) Applicant: Cilag GmbH International, Zug (CH)

(72) Inventors: Jacob Gee, Cincinnati, OH (US); Tyler Brehm, Cincinnati, OH (US); Scotty Chung, Cincinnati, OH (US)

(21) Appl. No.: 19/341,047

(22) Filed: Sep. 26, 2025

Related U.S. Application Data
(60) Provisional application No. 63/740,944, filed on Dec. 31, 2024.

(51) Int. Cl. A61B 18/12; A61B 18/00

(52) U.S. Cl. CPC A61B 18/12; A61B 2018/00404; A61B 2018/0063; A61B 2018/00642; A61B 2018/00666; A61B 2018/00708; A61B 2018/0072; A61B 2018/00755; A61B 2018/00767; A61B 2018/00779; A61B 2018/00791; A61B 2018/00845; A61B 2018/00886; A61B 2018/00994

(57) ABSTRACT

A control method for an energy-based surgical instrument includes activating energy at a subtherapeutic level, measuring a system response at the subtherapeutic level, adjusting one or more parameters based on the system response, and then activating energy at a therapeutic level according to the determined parameters. Tissue pad wear of the surgical instrument may be determined based on ultrasound usage of the instrument, and radio frequency (RF) energy output parameters may be determined based on the tissue pad wear. Attributes of an RF sealing operation may be recorded, and one or more control parameters of an ultrasonic transection operation may be determined based on the attributes of the RF sealing operation. Other embodiments are described and claimed.

### CROSS-REFERENCE TO RELATED APPLICATION

This application claims the benefit of and priority to U.S. Patent Application No. 63/740,944, entitled “TECHNOLOGIES FOR THERAPEUTIC AND SUBTHERAPEUTIC CONTROL OF ENERGY-BASED SURGICAL INSTRUMENTS,” which was filed on Dec. 31, 2024, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient's tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient's tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100 kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, a method for controlling a surgical instrument includes recording, by a control element, ultrasound usage of the surgical instrument; determining, by the control element, tissue pad wear of the surgical instrument based on the ultrasound usage of the surgical instrument; and determining, by the control element, a radio frequency (RF) energy output parameter for the surgical instrument based on the tissue pad wear. In some embodiments, the method further includes activating, by the control element, delivery of RF energy by the surgical instrument to tissue of a patient with the RF energy output parameter.

In some embodiments, recording the ultrasound usage of the surgical instrument comprises recording a total time of ultrasound activation. In some embodiments, recording the ultrasound usage of the surgical instrument comprises recording a number of ultrasound activations, a longest ultrasound activation time, or a number of ultrasound activations having a time longer than a predetermined threshold.

In some embodiments, determining the tissue pad wear includes determining a change in tissue pad height based on the ultrasound usage. In some embodiments, determining the RF energy output parameter includes reducing an output voltage of the RF energy based on the change in tissue pad height.

According to another aspect, a method for controlling a surgical instrument includes activating, by a control element, delivery of ultrasonic energy by the surgical instrument at a subtherapeutic level during a first phase; measuring, by the control element, a frequency shift of an ultrasonic blade of the surgical instrument in response to activating the delivery of the ultrasonic energy at the subtherapeutic level; determining, by the control element, a control parameter based on the frequency shift; and activating, by the control element, delivery of ultrasonic energy by the surgical instrument at a therapeutic level with the control parameter during a second phase after the first phase.

In some embodiments, the frequency shift comprises a change in resonant frequency of the ultrasonic blade compared to a predetermined resonant frequency of the ultrasonic blade at a predetermined cold temperature. In some embodiments, the control parameter comprises a current setpoint for the second phase.

In some embodiments, the first phase comprises an initial phase of the delivery of ultrasonic energy. Measuring the frequency shift includes measuring the frequency shift due to initial temperature of the ultrasonic blade.

In some embodiments, the method further includes determining, by the control element, whether an exit criterion has been reached based on the frequency shift; and stopping, by the control element, the delivery of the ultrasonic energy during the second phase in response to determining that the exit criterion has been reached. In some embodiments, the exit criterion comprises whether the frequency shift exceeds a predetermined threshold for a predetermined amount of time during the first phase. In some embodiments, the exit criterion comprises whether a first frequency shift at a beginning of the first phase is within a predetermined threshold of a second frequency shift at an end of the first phase.

According to another aspect, a method for controlling a surgical instrument includes activating, by a control element, a radio frequency (RF) seal operation with the surgical instrument; recording, by the control element, a first attribute of the RF seal operation; determining, by the control element, an ultrasonic control parameter based on the first attribute of the RF seal operation; and activating, by the control element, an ultrasonic transection operation with the surgical instrument using the ultrasonic control parameter.

In some embodiments, the first attribute of the RF seal operation comprises an RF seal operation duration, a termination impedance, or a rate of change of impedance. In some embodiments, the ultrasonic control parameter comprises ultrasonic blade tip displacement.

In some embodiments, determining the ultrasonic control parameter based on the first attribute of the RF seal operation includes determining a size of vessel based on the first attribute, and determining the ultrasonic control parameter based on the size of the vessel. In some embodiments, determining the ultrasonic control parameter based on the size of the vessel comprises increasing ultrasonic energy for smaller vessels and decreasing ultrasonic energy for larger vessels.

In some embodiments, determining the ultrasonic control parameter based on the first attribute of the RF seal operation includes determining a quality of seal based on the first attribute and determining the ultrasonic control parameter based on the quality of the seal. In some embodiments, determining the ultrasonic control parameter based on the quality of the seal comprises increasing ultrasonic energy for lower quality seals.

According to another aspect, a system for controlling a surgical instrument includes the surgical instrument and a control element. The surgical instrument includes an end effector having an ultrasonic blade configured to deliver ultrasonic energy to tissue of a patient, a tissue pad coupled to a jaw of the end effector and configured to clamp the tissue of the patient, and an electrode configured to deliver radio frequency (RF) energy to the tissue of a patient. The control element is configured to record ultrasound usage of the surgical instrument, determine tissue pad wear of the surgical instrument based on the ultrasound usage of the surgical instrument, and determine an RF energy output parameter for the surgical instrument based on the tissue pad wear. In some embodiments, the control element is further configured to activate delivery of RF energy by the surgical instrument to the tissue of the patient with the RF energy output parameter.

In some embodiments, to record the ultrasound usage of the surgical instrument includes to record a total time of ultrasound activation. In some embodiments, to record the ultrasound usage of the surgical instrument includes to record a number of ultrasound activations, a longest ultrasound activation time, or a number of ultrasound activations having a time longer than a predetermined threshold.

In some embodiments, to determine the tissue pad wear includes to determine a change in height of the tissue pad based on the ultrasound usage. In some embodiments, to determine the RF energy output parameter includes to reduce an output voltage of the RF energy based on the change in tissue pad height.

According to another aspect, a system for controlling a surgical instrument includes the surgical instrument and a control element. The surgical instrument includes an end effector having an ultrasonic blade configured to deliver ultrasonic energy to tissue of a patient. The control element is configured to activate delivery of ultrasonic energy by the surgical instrument at a subtherapeutic level during a first phase, measure a frequency shift of the ultrasonic blade in response to activation of the delivery of the ultrasonic energy at the subtherapeutic level, determine a control parameter based on the frequency shift, and activate delivery of ultrasonic energy by the surgical instrument at a therapeutic level with the control parameter during a second phase after the first phase.

In some embodiments, the frequency shift comprises a change in resonant frequency of the ultrasonic blade compared to a predetermined resonant frequency of the ultrasonic blade at a predetermined cold temperature. In some embodiments, the control parameter comprises a current setpoint for the second phase.

In some embodiments, the first phase comprises an initial phase of the delivery of ultrasonic energy. To measure the frequency shift includes to measure the frequency shift due to initial temperature of the ultrasonic blade.

In some embodiments, the control element is further configured to determine whether an exit criterion has been reached based on the frequency shift, and stop the delivery of the ultrasonic energy during the second phase in response to a determination that the exit criterion has been reached. In some embodiments, the exit criterion comprises whether the frequency shift exceeds a predetermined threshold for a predetermined amount of time during the first phase. In some embodiments, the exit criterion comprises whether a first frequency shift at a beginning of the first phase is within a predetermined threshold of a second frequency shift at an end of the first phase.

According to another aspect, a system for controlling a surgical instrument includes the surgical instrument and a control element. The surgical instrument includes an end effector having an ultrasonic blade configured to deliver ultrasonic energy to tissue of a patient and an electrode configured to deliver radio frequency (RF) energy to the tissue of a patient. The control element is configured to activate a radio frequency (RF) seal operation with the surgical instrument, record a first attribute of the RF seal operation, determine an ultrasonic control parameter based on the first attribute of the RF seal operation, and activate an ultrasonic transection operation with the surgical instrument using the ultrasonic control parameter.

In some embodiments, the first attribute of the RF seal operation comprises an RF seal operation duration, a termination impedance, or a rate of change of impedance. In some embodiments, the ultrasonic control parameter comprises ultrasonic blade tip displacement.

In some embodiments, to determine the ultrasonic control parameter based on the first attribute of the RF seal operation includes to determine a size of vessel based on the first attribute, and determine the ultrasonic control parameter based on the size of the vessel. In some embodiments, to determine the ultrasonic control parameter based on the size of the vessel includes to increase ultrasonic energy for smaller vessels and decrease ultrasonic energy for larger vessels.

In some embodiments, to determine the ultrasonic control parameter based on the first attribute of the RF seal operation includes to determine a quality of seal based on the first attribute and determine the ultrasonic control parameter based on the quality of the seal. In some embodiments, to determine the ultrasonic control parameter based on the quality of the seal includes to increase ultrasonic energy for lower quality seals.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a simplified flow diagram of at least one method for controlling an energy-based surgical instrument;

FIG. 9 is a simplified flow diagram of at least one method for controlling a combined radio frequency (RF) and ultrasonic energy-based surgical instrument;

FIG. 10 is a schematic diagram illustrating an end effector of an energy-based surgical instrument that may be used with the method of FIG. 9;

FIG. 11 is a simplified flow diagram of at least one method for controlling a ultrasound or combined energy-based surgical instrument;

FIG. 12 is a schematic diagram illustrating operation of a surgical instrument according to the method of FIG. 11; and

FIG. 13 is a simplified flow diagram of at least one method for controlling a combined energy-based surgical instrument.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient's natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

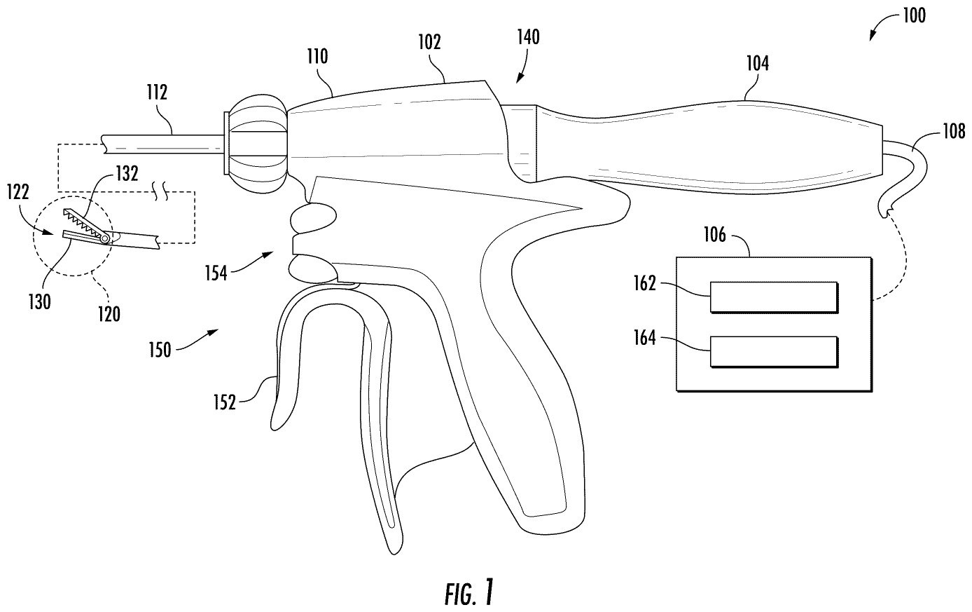

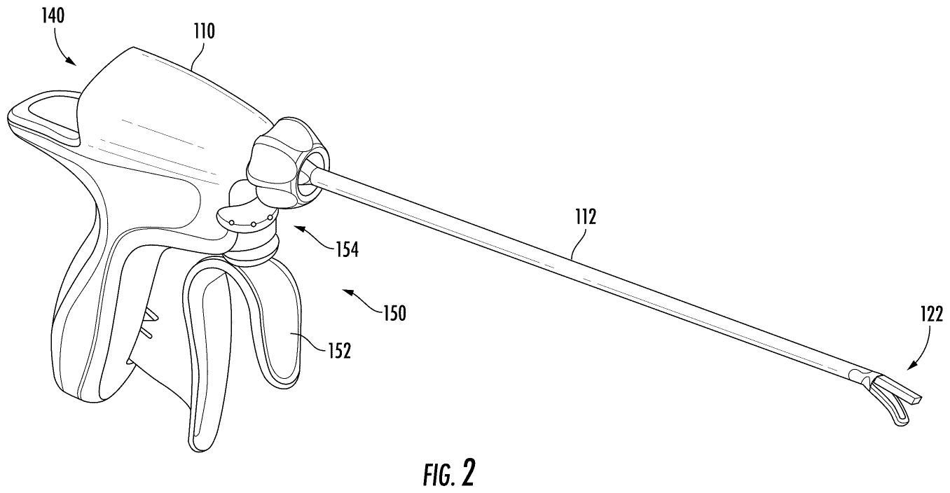

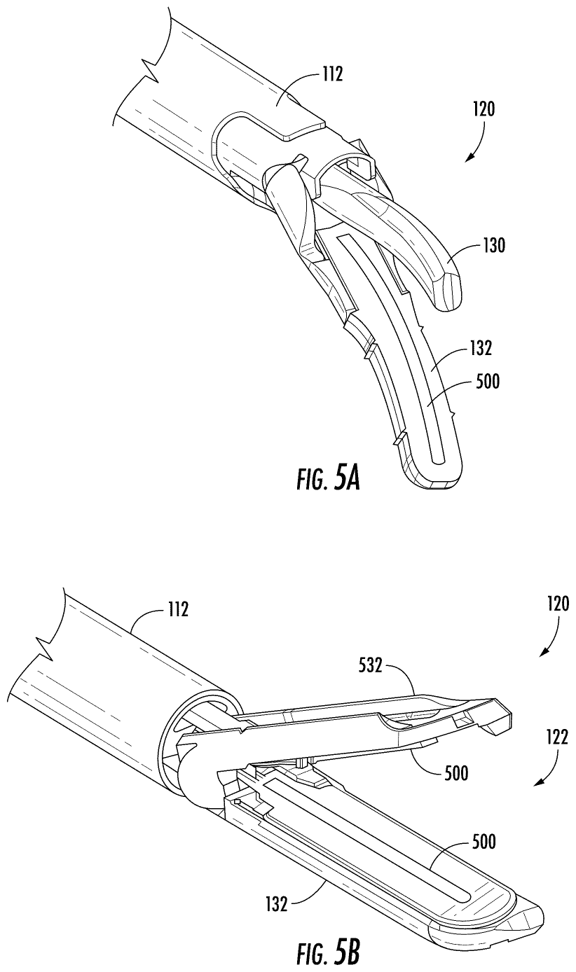

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

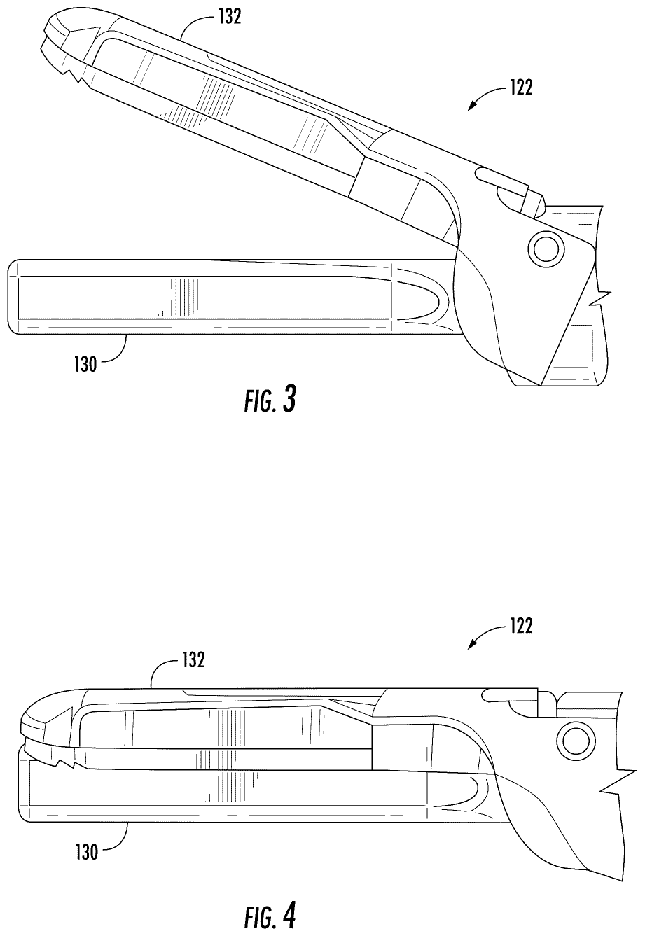

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both a ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient's body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient's tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient's tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient's tissue. For example, the electro-surgical generator module 164 may be configured to measure an impedance of the patient's tissue using the electrodes 500 and a suitable RF energy level.

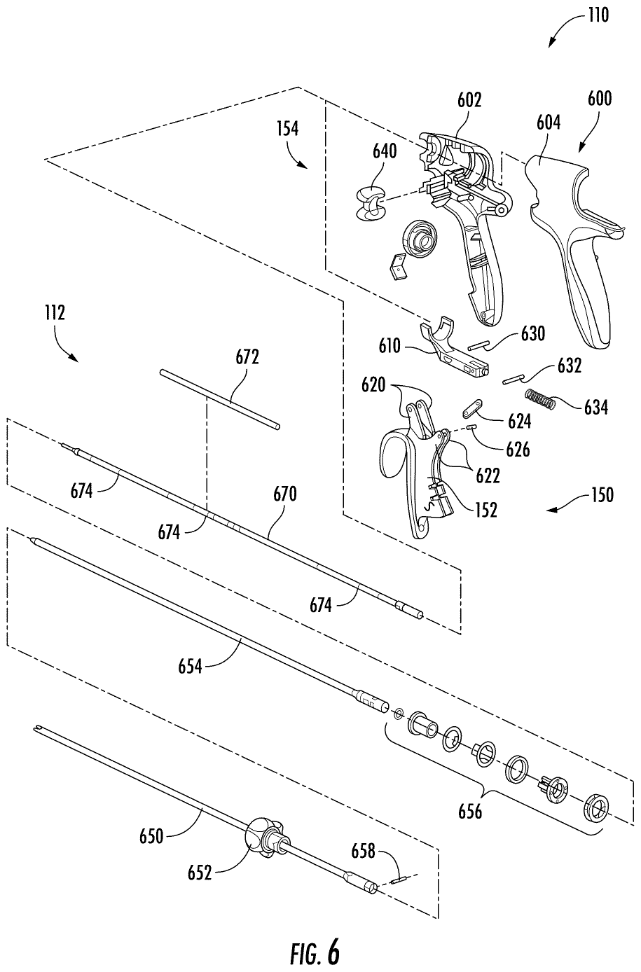

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

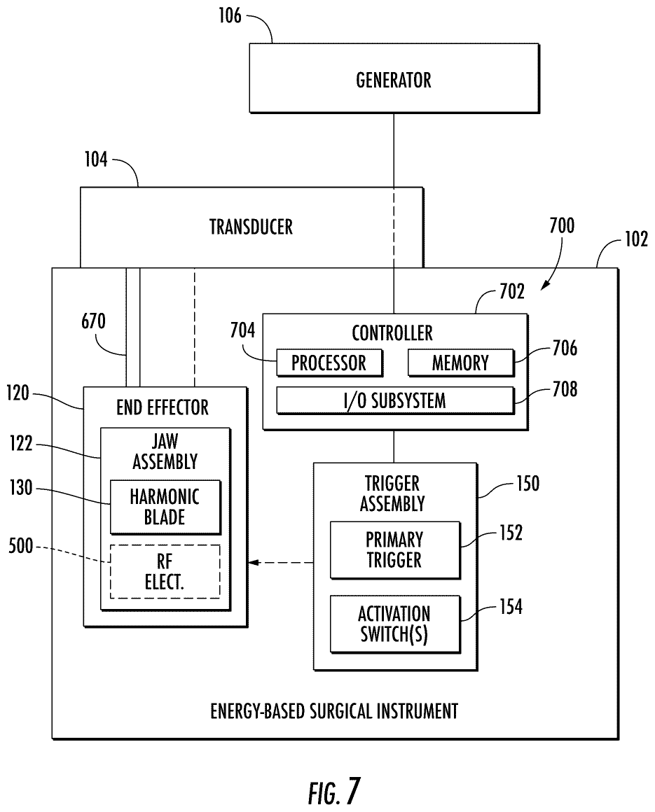

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

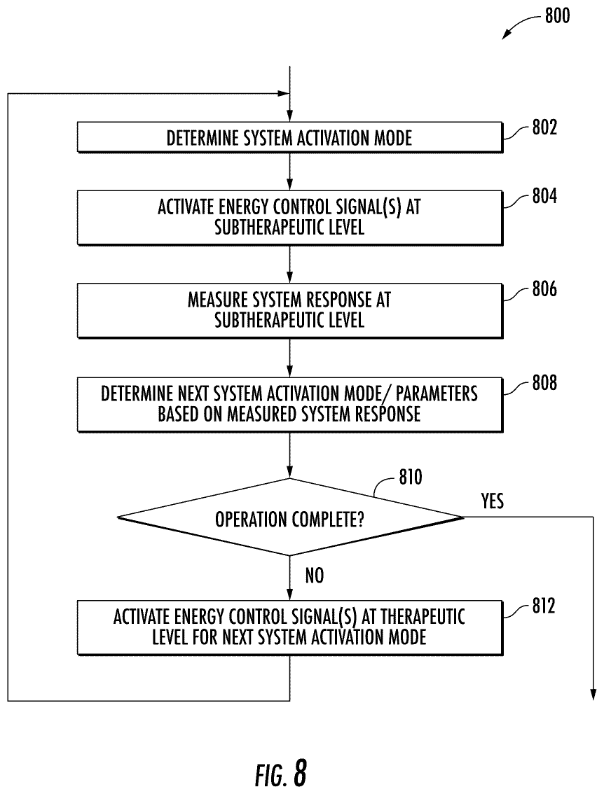

Referring now to FIG. 8, a method 800 for controlling an energy-based surgical instrument 102 is shown. The method 800 may be executed by the controller 702, the generator 106, and/or one or more other microcontrollers or other control elements of the system 100. The method 800 begins in block 802, in which the control element determines a system activation mode for the surgical instrument 102. The system activation mode may include an energy modality (e.g., RF, ultrasound, combined RF and ultrasound, etc.), a surgical operation or firing to be performed with the surgical instrument 102 (e.g., seal, transect, seal and transect, etc.), and/or a sub-operation or phase (e.g., heating, sensing, sealing, cutting, etc.).

In block 804, the control element activates one or more energy control signals at a subtherapeutic level. The subtherapeutic level may be a lower power or energy level that does not cause coagulation, transection, or other therapeutic actions in tissue. The subtherapeutic level may cause other responses in the tissue, such as subtherapeutic heating. The subtherapeutic control may activate ultrasound energy, RF energy, or combined ultrasound and RF energy at the subtherapeutic level.

In block 806, the control element measures a system response at the subtherapeutic level. For example, the control element may measure tissue impedance, acoustic impedance, frequency shift, phase shift, or other responses to application of the subtherapeutic signal.

In block 808, the control element determines a next system activation mode and/or parameters based on the measured system response. For example, the control element may determine whether to switch energy modalities (e.g., from RF to ultrasound, from ultrasound to RF, from a single modality to a combined modality, or other change in energy modality). As another example, the control element may determine whether to change sub-operation or phase, e.g., from pre-heating to sealing, from sealing to transecting, or other change in sub-operation. As another example, the control element may determine one or more parameters for application of therapeutic levels of energy, such as setpoint, amplitude, frequency, crest factor (CF), or other parameters. As yet another example, the control element may determine that the surgical operation (e.g., sealing and/or transecting tissue) has been completed.

In block 810, the control element checks whether the present surgical operation or firing has been completed. If so, the method 800 is completed. The method 800 may be executed again in response to subsequent surgical firings. If the surgical operation is not complete, the method 800 advances to block 812.

In block 812, the control element activates one or more energy control signals at a therapeutic level for the next system activation mode determined as described above. For example, the control element may activate ultrasound and/or RF energy at a setpoint determined as described above or otherwise cause activation of the surgical instrument 102. After activation, the method 800 may loop back to block 802 to continue performing subtherapeutic measurement and control of therapeutic energy application.

Additionally or alternatively, in some embodiments the control element may perform the operations of the method 800 in a different order and/or in a different combination. Further, in some embodiments the control element may perform additional or different operations and/or make additional or different measurements. Illustrative examples of control operations that may be performed in connection with the surgical instrument 102 are described further below in connection with FIGS. 9-20.

Referring now to FIG. 9, a method 900 for controlling a combined energy-based surgical instrument 102 is shown. The method 900 may be executed by the controller 702, the generator 106, and/or one or more other microcontrollers or other control elements of the system 100. The method 900 may be executed, for example, in connection with monopolar or bipolar electrosurgery using the surgical instrument 102. The method 900 begins in block 902, in which the control element determines a system activation mode for the surgical instrument 102. The system activation mode may include an energy modality (e.g., radio frequency (RF), ultrasound, and/or combined RF and ultrasound, etc.).

In block 904, the control element determines whether the system activation mode indicates ultrasonic operation. If not, the method 900 branches ahead to block 908. If the control element determines that the system activation mode includes ultrasonic operation, the method 900 advances to block 906.

In block 906, the control element records ultrasonic energy usage by the surgical instrument 102. The control element may record any one or more metrics or other indications of ultrasonic energy usage. For example, the control element may record total ultrasonic usage such as total activation time, total number of ultrasonic activations, longest ultrasonic activation times, a number of long ultrasonic activations (e.g., ultrasonic activations with length greater than a predetermined threshold), or other measures of ultrasonic usage. After recording ultrasonic usage, the method 900 advances to block 908.

In block 908, the control element whether the system activation mode indicates radio frequency (RF) operation. If not, the method 900 loops back to block 902, in which the control element continues to monitor system activation. If the control element determines that the system activation mode includes RF operation, the method 900 advances to block 910.

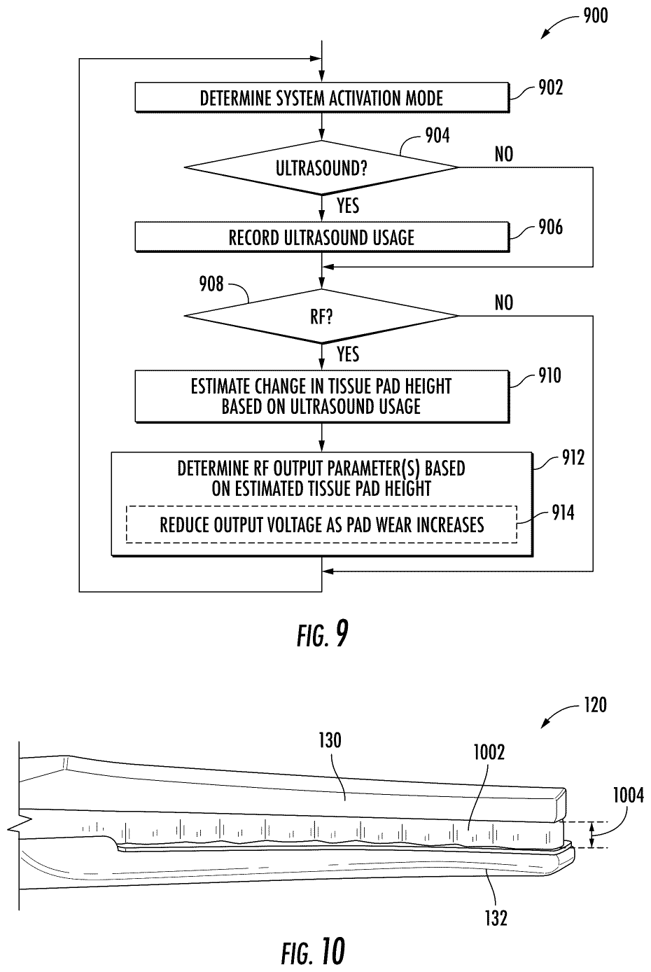

In block 910, the control element estimates a change in tissue pad height based on ultrasonic energy usage. As illustrated in FIG. 10 and discussed further below, the end effector 120 of the surgical instrument 102 includes a polymeric tissue pad that is eroded or otherwise worn away by the ultrasonic blade 130 during delivery of ultrasonic energy. This tissue pad wear results in a change in tissue pad height (i.e., the distance between blade 130 and electrode 500), which can be estimated/calculated by tracking the usage of ultrasonic energy. As described above, the tissue pad wear rate may be estimated/calculated using recorded data indicative of the total ultrasonic usage, such as total activation time, number of activations, the longest activations, the number of long activations, or other measures of tissue pad wear. may determine the tissue pad wear based on ultrasound usage.

In block 912, the control element adjusts or otherwise determines one or more RF output parameters based on the estimated change in tissue pad height (or other determination of tissue pad wear). The ideal RF output for a desired tissue type may be different based on the distance between the two RF poles, e.g., the distance between the harmonic blade 130 and the electrode 500. When this distance is large, more voltage may be needed than when the distance is small. Due to tissue variation, presence of fluid, and other factors, this electrode distance may not be determined by measuring RF impedance or other electrical parameters. Accordingly, one or more of the RF output parameters (e.g., voltage, current, crest factor (CF), or other output parameter) are altered based on the calculated pad wear rate as the surgical instrument 102 is used with ultrasonic energy. In some embodiments, in block 914 the control element reduces output voltage for the RF operation as pad wear increases.

After determining the RF output parameters, the method 900 loops back to block 902 to continue monitoring system activation. The surgical instrument 102 may perform subsequent RF operations using the adjusted RF output parameters, allowing RF output to vary as the tissue pad wears over use.

Referring now to FIG. 10, an illustrative end effector 120 of a surgical instrument 102 is shown. As shown, the end effector 1002 includes an ultrasonic blade 130 and a jaw clamp 132, which is coupled to an electrode 500 (not shown). A polymeric tissue pad 1002 is coupled to the jaw clamp 132. The tissue pad 1002 is illustratively formed from polytetrafluoroethylene (PTFE). Additionally or alternatively, the tissue pad 1002 may be formed from a different polymeric material, such as but not limited to polyetheretherketone (PEEK), polyetherimide (PEI), polyphenylene sulfide (PPS), polyimide (PI), polysulfone (PSU), polypropylene (PP), polystyrene (PS), polyethylene terephthalate (PET), fiberglass (FG), polycarbonate (PC), acrylonitrile butadiene styrene (ABS), and/or polyvinylchloride (PVC).

As shown, when the end effector jaws are clamped together, the blade 130 contacts the tissue pad 1002 and the blade 130 is separated from the jaw clamp 132 (and thus the electrode 500) by a tissue pad height 1004. As the surgical instrument is activated in ultrasonic energy mode, the blade 130 erodes the tissue pad 1002, which reduces the tissue pad height. As described above, the control element adjusts RF output parameters based on the estimated remaining tissue pad height 1004, which in turn is determined based on total ultrasonic activations of the surgical instrument 102.

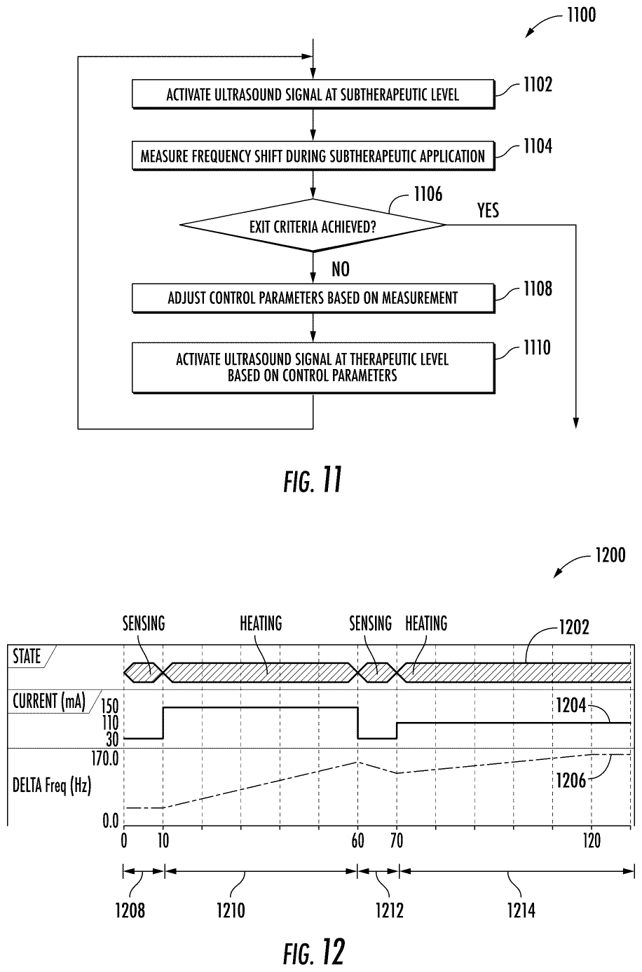

Referring now to FIG. 11, a method 1100 for controlling an energy-based surgical instrument 102 is shown. The method 1100 may be executed by the controller 702, the generator 106, and/or one or more other microcontrollers or other control elements of the system 100. The method 1100 may be executed, for example, in connection with ultrasound-only sealing using the surgical instrument 102. The method 1100 begins in block 1102, in which the control element activates an ultrasound control signal at a subtherapeutic level.

In block 1104, the control element measures frequency shift during application of subtherapeutic ultrasound energy. The measured frequency shift is the shift of resonant frequency of the ultrasound blade 130 from a cold temperature at manufacturing. The measured frequency shift is proportional to the temperature of the tip of the blade 130. An initial subtherapeutic segment may account for retained blade heat. For example, the initial subtherapeutic segment may measure frequency shift from the cold resonant frequency due to the initial temperature of the blade 130. When the frequency shift of the blade 130 exceeds a particular threshold or range for a predetermined amount of time, this may indicate that the tissue has been coagulated and/or cut. As another example, when the frequency shift of the blade 130 stabilizes during the subtherapeutic segment (e.g., the frequency shift is equal or nearly equal at the start and end of the subtherapeutic segment), this may indicate that the tissue has been coagulated and/or cut. Additionally or alternatively, when the frequency shift of the blade 130 falls below a particular threshold or range, this may indicate a blade failure or breakage or a tissue change.

In block 1106, the control element determines whether exit criteria have been achieved. Exit criteria may include, for example, whether a sufficient frequency shift (either positive or negative) has been achieved for a required duration. As another example, energy cycles may be applied until the frequency shift during the start and end of the subtherapeutic sensing period is nearly equal (i.e., the tissue sufficiently heated). As another example, the exit criteria may be based on the rate of change of frequency during the subtherapeutic sensing period. If the exit criteria have been achieved, the method 1100 is completed, and the surgical instrument 102 may stop applying ultrasound energy. If the exit criteria have not been achieved, the method 1100 advances to block 1108.

In block 1108, the control element adjusts one or more control parameters based on the measurement. For example, the control parameters may include current setpoint, blade tip displacement (power), or another indication of the amount of ultrasound energy to be applied during a therapeutic cycle. Accordingly, each therapeutic segment may be controlled according to measurements made in the previous subtherapeutic segment. For example, based on the initial condition of the blade (i.e., initial temperature of blade 130), the control element may determine how much energy to deliver on the next therapeutic cycle.

In block 1110, the control element activates an ultrasound control signal at a therapeutic level based on the determined one or more control parameters. For example, the control element may activate ultrasonic energy delivery to the blade 130 at the current setpoint determined as described above. After activating the therapeutic level of ultrasound energy, the method 1100 loops back to block 1102 to perform additional subtherapeutic sensing.

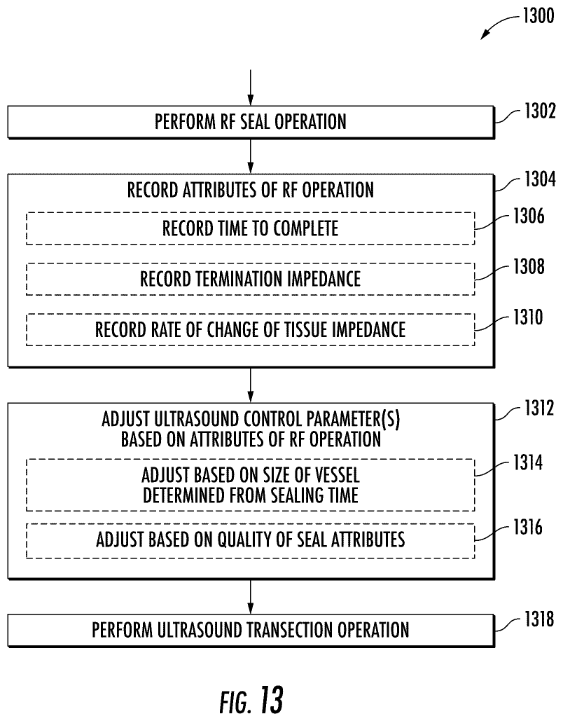

Referring now to FIG. 12, diagram 1200 illustrates operation of a surgical instrument 102 with subtherapeutic sensing. Trace 1202 illustrates the state of the surgical instrument. Curve 1204 illustrates current applied by the generator 106 to the ultrasonic transducer 104. Curve 1206 illustrates the change in frequency of the ultrasound energy, which is measured during the subtherapeutic sensing states. Accordingly, using ultrasound energy only, the instrument 102 switches between therapeutic levels and subtherapeutic levels to achieve a seal-only effect.

In the illustrative embodiment, in an initial subtherapeutic phase 1208, the control element measures frequency shift due to the initial temperature of the blade 130 and determines a current setpoint. In therapeutic phase 1210, the control element applies the therapeutic ultrasonic energy to the blade 130 at the determined current setpoint. As shown, the frequency shift increases during the phase 1210, indicating increasing temperature of the blade 130 and thus of the tissue. In subtherapeutic phase 1212, the control element measures frequency shift and determines an updated current setpoint. The control element may also determine that an exit criterion has not been satisfied; for example, the control element may determine that the frequency shift has not yet reached a threshold amount, or that the frequency shift is not equal (or nearly equal) at the beginning and end of the phase 1212. In therapeutic phase 1214, the control element applies the therapeutic ultrasonic energy to the blade 130 at the updated current setpoint. As shown, the current setpoint in the therapeutic phase 1214 is lower than the current setpoint in the therapeutic phase 1210, illustratively due to the increased frequency shift (and thus increased temperature) of the blade 130. As also shown, at the end of the therapeutic phase 1214, the frequency shift stabilizes, indicating that sealing has been achieved. Accordingly, the control element may stop the sealing operation subsequent to the phase 1214.

As shown, by performing subtherapeutic sensing, current applied during different therapeutic cycles may be varied according to the condition of the surgical instrument (e.g., heat retained in the blade 130). Further, the seal-only operation may exit early when exit criteria are satisfied, as compared to traditional time-based approaches that applied the same sealing time to smaller and larger vessels. Additionally, former time-based approaches may transect through tissue after sealing. As described above, the illustrative technique may exit upon satisfaction of one or more exit criteria, meaning that the surgical instrument 102 may perform a seal-only (hemostasis) operation, without final transection of the tissue.

Referring now to FIG. 13, a method 1300 for controlling an energy-based surgical instrument 102 is shown. The method 1300 may be executed by the controller 702, the generator 106, and/or one or more other microcontrollers or other control elements of the system 100. The method 1300 may be executed, for example, in connection with combined RF and ultrasound operation of the surgical instrument 102. The method 1300 begins in block 1302, in which the control element causes the surgical instrument 102 to perform an RF seal operation. As described above, during the RF seal operation, one or more electrodes 500 of the surgical instrument 102 delivers therapeutic RF energy to tissue of the patient, such as one or more blood vessels, in a coagulation or sealing operation. The RF sealing operation may continue until one or more exit criteria are met, such as a predetermined elapsed time or amount of RF energy, or a predetermined response such as measured electrical impedance of the patient's tissue.

In block 1304, the control element measures and/or records one or more attributes of the RF sealing operation, such as duration, tissue impedance, or other parameters. In some embodiments, in block 1306 the control element records time to complete the RF sealing operation. In some embodiments, in block 1308 the control element records termination impedance, which is the electrical impedance of the tissue at the time the RF sealing operation is completed. In some embodiments, in block 1310 the control element records a rate of change of tissue impedance.

In block 1312, the control element adjusts one or more ultrasound control parameters based on the measured attributes(s) of the previous RF sealing operation. The ultrasound control parameters may include blade 130 displacement (i.e., ultrasonic power). In some embodiments, in block 1314, the control element may adjust an ultrasonic blade 130 displacement (i.e., power) based on a blood vessel size determined from the RF seal operation duration. For example, lower blade displacements may be utilized following an RF seal that took a longer duration, as this is an indication that the vessel being sealed is larger and the ultrasound energy should be lower to not damage the seal and actually improve the seal. Conversely, if the RF activation is quick, the vessel is smaller and the ultrasound energy can be higher to cut through the vessel faster.

In some embodiments, in block 1316, the control element may adjust the ultrasound control parameters based on seal quality determined from the recorded attributes of the RF sealing operation. For example, the control element may determine seal quality based on termination impedance, impedance rate of change during the previous RF sealing operation, or other information about the RF seal. The control element may determine, for example, if the seal was good, marginal, or bad. For example, if the termination impedance is relatively high, then the tissue was likely completely sealed. The control element uses this information about the RF seal quality to determine the amount of ultrasound power to be delivered to ensure the vessel/tissue is sealed and transected with the additional ultrasound energy being delivered after the RF seal. For example, in an illustrative embodiment, if the control element determines that the seal quality is marginal or bad, the control element may increase ultrasonic energy in order to ensure complete transection and sealing. Of course, other adjustments to ultrasonic energy may be performed in other embodiments.

In block 1318, the control element causes the surgical instrument 102 to perform ultrasound transection operation with the determined ultrasound control parameters. For example, the surgical instrument 102 may perform the ultrasonic operation with blade displacement determined based on attributes of the previous RF sealing operation, as described above. Accordingly, the amount of ultrasound power (blade displacement) is “smart” and reactive to the information about the RF seal that just happened.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A method for controlling a surgical instrument, the method comprising:
recording, by a control element, ultrasound usage of the surgical instrument;
determining, by the control element, tissue pad wear of the surgical instrument based on the ultrasound usage of the surgical instrument; and
determining, by the control element, a radio frequency (RF) energy output parameter for the surgical instrument based on the tissue pad wear.

2. The method of claim 1, further comprising activating, by the control element, delivery of RF energy by the surgical instrument to tissue of a patient with the RF energy output parameter.

3. The method of claim 1, wherein recording the ultrasound usage of the surgical instrument comprises recording a total time of ultrasound activation.

4. The method of claim 1, wherein recording the ultrasound usage of the surgical instrument comprises recording a number of ultrasound activations, a longest ultrasound activation time, or a number of ultrasound activations having a time longer than a predetermined threshold.

5. The method of claim 1, wherein determining the tissue pad wear comprises determining a change in tissue pad height based on the ultrasound usage.

6. The method of claim 5, wherein determining the RF energy output parameter comprises reducing an output voltage of the RF energy based on the change in tissue pad height.

7. A method for controlling a surgical instrument, the method comprising:
activating, by a control element, delivery of ultrasonic energy by the surgical instrument at a subtherapeutic level during a first phase;
measuring, by the control element, a frequency shift of an ultrasonic blade of the surgical instrument in response to activating the delivery of the ultrasonic energy at the subtherapeutic level;
determining, by the control element, a control parameter based on the frequency shift; and
activating, by the control element, delivery of ultrasonic energy by the surgical instrument at a therapeutic level with the control parameter during a second phase after the first phase.

8. The method of claim 7, wherein the frequency shift comprises a change in resonant frequency of the ultrasonic blade compared to a predetermined resonant frequency of the ultrasonic blade at a predetermined cold temperature.

9. The method of claim 7, wherein the control parameter comprises a current setpoint for the second phase.

10. The method of claim 7, wherein the first phase comprises an initial phase of the delivery of ultrasonic energy, and wherein measuring the frequency shift comprises measuring the frequency shift due to initial temperature of the ultrasonic blade.

11. The method of claim 7, further comprising:
determining, by the control element, whether an exit criterion has been reached based on the frequency shift; and
stopping, by the control element, the delivery of the ultrasonic energy during the second phase in response to determining that the exit criterion has been reached.

12. The method of claim 11, wherein the exit criterion comprises whether the frequency shift exceeds a predetermined threshold for a predetermined amount of time during the first phase.

13. The method of claim 11, wherein the exit criterion comprises whether a first frequency shift at a beginning of the first phase is within a predetermined threshold of a second frequency shift at an end of the first phase.

14. A method for controlling a surgical instrument, the method comprising:
activating, by a control element, a radio frequency (RF) seal operation with the surgical instrument;
recording, by the control element, a first attribute of the RF seal operation;
determining, by the control element, an ultrasonic control parameter based on the first attribute of the RF seal operation; and
activating, by the control element, an ultrasonic transection operation with the surgical instrument using the ultrasonic control parameter.

15. The method of claim 14, wherein the first attribute of the RF seal operation comprises an RF seal operation duration, a termination impedance, or a rate of change of impedance.

16. The method of claim 14, wherein the ultrasonic control parameter comprises ultrasonic blade tip displacement.

17. The method of claim 14, wherein determining the ultrasonic control parameter based on the first attribute of the RF seal operation comprises (i) determining a size of vessel based on the first attribute, and (ii) determining the ultrasonic control parameter based on the size of the vessel.

18. The method of claim 17, wherein determining the ultrasonic control parameter based on the size of the vessel comprises increasing ultrasonic energy for smaller vessels and decreasing ultrasonic energy for larger vessels.

19. The method of claim 14, wherein determining the ultrasonic control parameter based on the first attribute of the RF seal operation comprises (i) determining a quality of seal based on the first attribute and (ii) determining the ultrasonic control parameter based on the quality of the seal.

20. The method of claim 19, wherein determining the ultrasonic control parameter based on the quality of the seal comprises increasing ultrasonic energy for lower quality seals.
